(19) United States
(12) Patent Application Publication
(10) Pub. No.: US 2023/0405071 A1
(43) Pub. Date: Dec. 21, 2023

(54) COMPOSITIONS AND METHODS USEFUL FOR MANAGEMENT OF BLOOD SUGAR

(71) Applicant: SOLVEAT FUNCTIONAL NUTRITION SOLUTIONS LTD., Tel Aviv (IL)

(72) Inventors: Tal NAVEH, Tel Aviv (IL); Udi PERETZ, Tel Aviv (IL); Xia Long JIANG, Sichuan (CN); Zakhar NUDELMAN, Netanya (IL)

(21) Appl. No.: 18/034,737

(22) PCT Filed: Oct. 28, 2021
(86) PCT No.: PCT/IL2021/051280

§ 371 Date: May 1, 2023

Related U.S. Application Data
(60) Provisional application No. 63/108,374, filed on Nov. 1, 2020.

(51) Int. Cl. A61K 36/61; A61K 36/8984; A61K 36/428; A61K 36/718; A61P 3/10

(52) U.S. Cl. CPC A61K 36/61; A61K 36/8984; A61K 36/428; A61K 36/718; A61P 3/10

(57) ABSTRACT

Provided herein a composition comprising traditional Chinese remedies, as a new formula useful to treating or prophylaxis of hyperglycemia in a patient in need thereof.

### FIELD OF THE INVENTION

The invention relates to the field of health care and more specifically, to compositions and methods, the compositions and methods useful for treating conditions caused or characterized by elevated blood glucose concentrations. Particularly, compositions comprising combinations of traditional Chinese remedies are disclosed.

### BACKGROUND

Elevated blood glucose concentrations, also known as “high sugar” or “excess blood sugar” in popular culture, is considered a medical problem. Humans and non-human mammals may be disposed to hyperglycemic symptoms, resulting from a variety of underlying causes. Various ailments can express hyperglycemia, for example, diabetes mellitus, particularly type 2 diabetes, metabolic syndrome, pre-diabetic conditions (e.g. glucose intolerance), pregnancy-associated hyperglycemia, and others. Prolonged hyperglycemia may result in a variety of complications, morbidities, and ultimately death.

Due to the prevalence and severity of the condition, hyperglycemia has been recognized since the dawn of history in various cultures, and various approaches have been attempted to manage hyperglycemia and diabetes. In the Chinese classical medicine the conditions generally corresponds to the “wasting and thirsting disorder”, or xiao ke ( [symbol] ). The Nei Jing (Inner Classic), the pre-eminent classic of Chinese medicine, that was compiled in either the Spring and Autumn or Warring States period, mentions the name xiao ke, where it first appears in the Nei Jing where there is mention to several different though related conditions: xiao ke, wasting and thirsting, xiao dan, pure heat wasting, ge xiao, diaphragm wasting, and xiao zhong, central wasting. References in the Nei Jing to wasting and thirsting are scattered through 14 juan or books of this classic which discuss its disease causes and mechanisms, clinical manifestations, and treatment. In terms of the treatment of this disease, it was believed at this time that people with wasting and thirsting should eat and be treated by things which are sweet in flavor and cold in nature. This was believed to enable the engenderment of fluids and thus stop thirst. However, one should also have not eaten fatty, rich foods, used penetrating, aromatic herbs, or taken mineral medicinals which are dry and hot and damage fluids, based on Su Wen, “Treatise on the Abdomen & Center,” which teaches “[for] heat in the center/center wasting, it is not good to administer rich, fatty [foods], penetrating herbs, or stone medicinals.” In terms of disease causes, the authors of the Nei Jing recognized that overeating of sweets and fats, emotional stress, weakness of the five viscera, and obesity are all closely related to this disease. For instance, the Su Wen of Nei Jing (Essential Questions), “Treatise on Strange Diseases,” says: “[t]his [condition] occurs in those who are fat and beautiful (this disease occurs in bad eating habits). This person must [eat] many sweet, fine [foods] and too many fats. Fats all cause heat inside humans, and sweets all cause center fullness. Therefore, the qi spills over above, transforming into wasting and thirsting.” Additionally, the Ling Shu (Spiritual Axis), “The Five Changes,” teaches: “Anger leads the qi to counterflow upward where it amasses and accumulates in the center of the chest. The qi and blood [thus] counterflow and lodge, and the hip skin [i.e., fat] fills the muscles. The blood vessels do not move, and this transforms to make heat. Heat [then] leads to wasting of the muscles and skin. Therefore, this is called pure heat wasting.”. It also says, “[if] the five viscera are all soft and weak, there is the susceptibility to the disease of pure heat wasting.” While the Su Wen, “The Treatise on Understanding the Appraisal of Vacuity & Repletion,” says, “[in] attack of pure heat wasting, [being] fat and [eating] rich foods lead to the accumulation of fat [meats] and fine [foods].” The Ling Shu, “Evil Qi, the Viscera & Bowels, and Disease & Form,” speaking in terms of the heart, liver, spleen, lung, and kidney pulses says, “Faint and small (weak) makes for pure heat wasting.” Additionally, the Su Wen, “Treatise on the Living Qi Communicating with Heaven,” says, “[t]he changes of rich, fatty [foods are] the engenderment of large clove sores (malignant furuncle) on the feet.” In the late Han dynasty, Zhang Zhong-jing, in his Jin Gui Yao Lue (Essentials of the Golden Cabinet), also wrote about thirsting and wasting. According to Zhang, its main disease mechanisms are stomach heat and kidney vacuity (deficiency): “[if] yang floats, the pulse is floating and rapid. Floating refers to the qi, [while] rapidity refers to the dispersion of grains. [If the pulse is] also large and hard, [this is because] qi exuberance has led to many urinations. [If] the pulse is tight, the person should have many urinations. [If] the pulse is tight and fast, the person should suffer from the disease as wasting and thirsting. [If] the fu yang [or tarsal] pulse is rapid, the stomach has heat within it. This will lead to overheating and hard stool, and urination is numerous. [If] a man has wasting and thirsting, urination is contrarily numerous. [If] he drinks a bucket of water, he urinates a bucket of water.”

In traditional Chinese medicine diabetes symptoms have been treated with variety of herbal remedies for centuries, with varying success. Some evidence of the efficacy of such herbal remedies has been summarized, inter alia, in the review by Subhuti Dharmananda (Treatment of Diabetes with Chinese Herbs and Acupuncture, Internet Journal of the Institute for Traditional Medicine and Preventive Health Care, published on January 2003, available at http://www.itmonline.org/journal/arts/diabetes.htm, last accessed on 25 Oct. 2020).

Below presented is the list of the most commonly used herbs for the treatment of diabetes, according to Dharmananda. As used herein, the botanical names are given in abbreviated or full Linneic terminology, as the case may be, and the traditional Chinese name given in parentheses represented by atonal pinyin, and sometimes the traditional Chinese name is given in traditional hieroglyphic writing. Traditionally, diabetes has been associated with “wasting and thirsting disorder” (xiao ke). The Chinese traditional remedies suitable for the management of diabetes symptoms, include, according to Dharmananda, Alisma (ze xie), Anemarrhena (zhi mu), Astragalus (huang qi), Atractylodes (bai zhu), Dioscorea (shan yao), Ginseng (ren shen) Radix polygoni multiflora (he shou wu), Hoelen (fu ling), Lycium bark (di gu pi), Lycium (gou qi zi), Platycodon (jie geng), Rizoma polygonatum (huang jing), Radix pueraria (ge gen), Rehmannia (Sheng di huang), Radix salviae miltiorrhizae (dan shen), Scrophularia (xuan shen), Trichosanthes (tian hua fen), and Rhizoma polygonati odorati (yu zhu). These are used in the traditional formulas “Bai Hu Jia Ren Shen Tang” (BHJRT—white tiger decoction with ginseng), “Liuwei Dihuang Wan” (LDW—six-ingredient pill with Rehmannia), “Bawei Dihuang Wan” (BWH—eight-ingredient pill with Rehmannia), “Mai men dong Yin Zi” (MDYZ—Ophiopogon and Trichosanthes pill), “Fang Feng Tong Sheng San” (FFTSS—Saposhnikovia powder that sagely unblocks), “Yu Quan Wan” (YQW—Jade spring pill), and “Xiao Ke Wan”, which technically cannot be considered a traditional formula, as it contains sulfonylurea drug glibenclamide.

Additionally, according to “Chinese Herbal Medicine—Formulas & Strategies”, compiled and translated by Dan Bensky & Randall Barolet, first edition 1990 (ISBN 10: 0939616106/ISBN 13: 9780939616107), the symptoms of diabetes pertain to qi level of four-level system of differentiation, and are treatable by formulas that clear heat from qi level, such as White Tiger Decoction (bai hu tang) or Clear the Stomach Heat Powder (qing wei san). The compositions of these and other formulas are summarized in the table A.

TABLE A

| Ingredient | BHT | BHJRT | LDW | BDW | MDYZ | FFTSS | YQW | QWS |
|---|---|---|---|---|---|---|---|---|
| bai shao | | | | | | V | | |
| bai zhu | | | | | | V | | |
| bo he | | | | | | V | | |
| chuan xiong | | | | | | V | | |
| da huang | | | | | | V | | |
| dan zhu ye | | | | | V | | | |
| dang gui | | | | | | V | | |
| dang gui shen | | | | | | | | V |
| fang feng | | | | | | V | | |
| fu ling | | | V | V | V | | V | |
| gan cao | | | | | V | V | V | |
| ge gen | | | | | V | | V | |
| geng mi | V | V | | | | | | |
| hua shi | | | | | | V | | |
| huang lian | | | | | | | | V |
| huang qi | | | | | | | V | |
| huang qin | | | | | | V | | |
| jie geng | | | | | | V | | |
| jing jie | | | | | | V | | |
| lian qiao | | | | | | V | | |
| ma huang | | | | | | V | | |
| mai men dong | | | | | V | | V | |
| mang xiao | | | | | | V | | |
| mu dan pi | | | V | V | | | | V |
| ren shen | | V | | | V | | V | |
| shan yao | | | V | V | | | | |
| shan zhu yu | | | V | V | | | | |
| sheng di huang | | | V | V | V | | | V |
| sheng ma | | | | | | | | V |
| shi gao | V | V | | | | V | | |
| tian hua fen | | | | | V | | V | |
| wu mei | | | | | | | V | |
| wu wei zi | | | | V | V | | | |
| ze xie | | | V | V | | | | |
| zhi gan cao | V | V | | | | | | |
| zhi huang qi | | | | V | | | | |
| zhi mu | V | V | | | V | | | |
| zhi zi | | | | | | V | | |

In the table A, the terms used are as follows: bai shao (Paeoniae Radix alba), bai zhu
(Radix Atractylodes Macrocephalae), bo he (Lilii Bulbus Mentha Piperita Leaf), chuan
xiong (Rhizoma Ligustici Chuanxiong), da huang (radix et rhizoma rhei), dan zhu ye
(Lophatheri rhizoma et folii), dang gui (Radix Angelicae Sinensis), dang gui shen
(Radix Angelicae), fang feng (saposhnikoviae radix), fu ling (poria, hoelen), gan cao
(radix Glycyrrhizae Uralensis), ge gen (radix puerariae), geng mi (nonglutinous rice), hua
shi (talc), huang lian (Rhizoma Coptidis), huang qi (Astragalus), huang qin
(Radix Scutellariae Baicalensis), jing jie (Herba seu Flos Schizonepetae Tenuifoliae),
jie geng (Platycodon), lian qiao (Forsythiae Fructus), ma huang (ephedrae herba), mai men
dong - (ophiopogon tuber), mang xiao (natrii sulfas), mu dan pi (moutan cortex), ren shen -
(Ginseng radix), shan yao (dioscoreae rhizome), shan zhu yu (corni fructus), sheng di
huang (radix Rehmanniae Glutinosae), sheng ma (Rhizoma Cimicifugae), shi gao
(gypsum), tian hua feng - (radix trichosanthes), wu mei (Mume fructus), wu wei zi
(schisandrae fructus), ze xie (alismatis rhizome), zhi huang qi (astragali radix - honey prepared), zhi gan cao (honey roasted radix Glycyrrhizae Uralensis), zhi mu (radix Anemarrhena Asphodeloides), and zhi zi (fructus gardeniae).

These formulae are known for their multiple modifications. For example, Ba Hu Tang and Qing Wei San can be modified with a plethora of components, and, inter alia, with the following herbs below: with Radix et Rhizoma rhei (da huang), for a variety of indications unrelated to hyperglycemia, and with Herba dendrobii (shi hu), as part of BHT modified with 8 further herbs or minerals, for diabetes with thirst (e.g. as can be readily seen on the commercial website of Dr. Joel Penner https://www.americandragon.com/, last accessed on 27 Apr. 2021, for BHT on https://www.americandragon.com/Herb%20Formulas%20copy/BaiHuTang.html, and for QWS on https://www.americandragon.com/Herb%20Formulas%20copy/QingWeiSan.html, respectively). Other formulae and modifications are also known, as shown in the same website, and discussed in the review article “Traditional Chinese Medicines in Treatment of Patients with Type 2 Diabetes Mellitus”, Evid Based Complement Alternat Med. 2011; 2011: 726723, doi: by Weidong Xie et al.

For diabetes and related conditions, BHT and QWS can be modified as demonstrated in the table B below.

Interestingly, Bensky formulary also makes a remark about the Ginseng-modified Bai Hu Tang, citing the traditional literature, that “ . . . [i]t has been argued that [BHJRT] can be used when formulae relying on sheng di huang (Radix rehmanniae glutinosae) are ineffective . . . ”, indicating that modified BHT is considered a more effective treatment.

It is evident from the tables A and B, that despite the variety of available traditional Chinese remedies, it is desirable to have an improved composition that would be effective in controlling blood glucose concentrations. It is equally desirable to have a method for the management, e.g. treatment or prevention, of hyperglycemia. For example, it is desirable to have a composition and/or method for use in treatment of high blood sugar, e.g. by directly reducing its level, or by treating the underlying pathology that leads to the high blood sugar level.

TABLE B

| | BHT | | | | | QWS | |
|---|---|---|---|---|---|---|---|
| | | | Diabetes with | Diabetes with | Heat at Bi | | With |
| | | Modification | fatigue and | thirst, drinking, | and Qi with | | strong |
| Ingredient | Original | for Diabetes | cold sweat | and urination | high fever | Original | thirst |
| da huang | | | | | V | | |
| dang gui | | | | | | V | X |
| geng mi | V | X | X | X | V | | |
| hu zhang | | | | | V | | |
| huang bai | | | | | V | | |
| huang lian | | V | V | V | V | V | V |
| huang qin | | V | V | V | | | |
| mai men dong | | V | V | V | | | |
| mang xiao | | | | | V | | |
| mu dan pi | | | | | | V | V |
| ren shen | | | V | | | | |
| sheng di huang | | V | V | V | | V | V |
| sheng ma | | | | | | V | V |
| shi hu | | | | V | | | |
| shi gao | V | V | V | V | V | O | |
| tian hua fen | | V | V | | | | V |
| tian men dong | | V | V | V | | | |
| xuan shen | | | | | | | V |
| wu wei zi | | | | V | | | |
| zhi gan cao | V | V | V | V | V | | |
| zhi mu | V | V | V | V | V | | |
| zhi zi | | | | | V | | |

### SUMMARY OF THE INVENTION

In one aspect, provided herein a composition comprising a combination of several traditional Chinese remedies, e.g. herbal and/or mineral materials. The composition may be useful for the management of hyperglycemia and in conditions and pathologies conductive to hyperglycemia. In another aspect provided herein methods of making the compositions of the remedies, as well as method of managing, reducing the incidence, or preventing hyperglycemia with these compositions, and their use for the same purposes, as a further aspect.

The compositions may be useful for long-term management of hyperglycemia and symptoms thereof. The compositions preferably comprise the following traditional Chinese remedies' ingredients:
i) Psidium guajava L. ( [symbol] , fan shi liu ye),
ii) Herba dendrobii ( [symbol] , shi hu) and/or Mume fructus ( [symbol] , wu mei),
iii) Radix trichosanthis kirilowii ( [symbol] , tian hua fen) and/or Gypsum ( [symbol] , shi gao), and
iv) at least two of Rhizoma coptidis ( [symbol] , huang lian), Radix rehmanniae glutinosae ( [symbol] , sheng di huang), and Radix et Rhizoma rhei ( [symbol] , da huang).
   However, it should be noted that when either the component iii) or iv) comprises all its ingredients, the other component is optional; i.e. when the composition comprises tian hua fen and shi gao, neither huang lian, sheng di huang, nor da huang is required, and conversely, when the composition contains huang lian, sheng di huang, and da huang, no tian hua fen nor shi gao is required. Usually, these ingredients above comprise between 60 and 100% by weight of the composition. Optionally, both ingredients of the component ii) is present, i.e. shi hu and wu mei. Optionally, all three components of iv) huang lian, sheng di huang, and da huang are present. Further optionally, both components of iii) shi gao and tian hua fen are present. Preferably, the composition comprises all eight components fan shi lui ye, wu mei, shi hu, tian hua fen, shi gao, huang lian, sheng di huang, and da huang (herein: “the eight ingredients”).

The composition may further comprise Siraitia grosvenorii ( [symbol] , luo han guo), or any one of Rhizoma anemarrhenae asphodeloidis ( [symbol] , zhi mu), Cortex moutan radicis ( [symbol] , mu dan pi), Crataegi fructus ( [symbol] , shan zha), Penthorum chinense ( [symbol] , gan huang cao), and Silybum marianum ( [symbol] , shui fei ji). In some currently preferred embodiments, the composition comprises between 7 and 14% wt huang lian, between 4 and 12% wt sheng di huang, between 2 and 8% wt da huang, between 4 and 12% wt shi hu, between 2 and 8% wt shi gao, between 7 and 14% wt tian hua fen, between 7 and 14% wt wu mei, and between 37 and 45% wt fan shi liu ye. In some currently further preferred embodiments the composition comprises between 1 and 15% wt huang lian, between 5 and 40% wt sheng di huang, between 2 and 15% wt da huang, between 4 and 20% wt shi hu, between 2 and 30% wt shi gao, between 5 and 35% wt tian hua fen, between 5 and 20% wt wu mei, between 5 and 50% wt fan shi liu ye, between 5 and 30% wt zhi mu, between 3 and 20% wt mu dan pi, between 4 and 20% wt shan zha, between 5 and 30% wt gan huang cao, and between 3 and 30% wt shui fen ji.

The ingredients of the composition may be provided in a variety of forms according to the need, e.g. according to traditional Chinese compounding practice, or as an aqueous extract, or as an organic extract, or as a standardized extract, or as a tincture, or as a dried extract, or as any combination of these forms for one or more of the components.

The compositions according to the invention may be used for treating of a condition characterized by hyperglycemia, e.g. pre-diabetes, diabetes, gestational diabetes, glucose intolerance, metabolic syndrome, or xiao ke. Usually, treating comprises administering to a patient in need thereof of the composition in a dose of between 500 mg and 1500 mg, at a regimen of between once daily and four times daily.

Preferably, the composition is provided in a form of a comestible article, preferably as portioned edible article, such as of chocolate pre-split bars, chocolate cushions, individually wrapped candy, energy bars, cookies, cake portions, crackers, pastilles, gums, and films.

In a further aspect, provided herein a method of manufacturing of a composition suitable for use in treating a condition associated with hyperglycemia, said method comprising providing ingredients comprising botanical or mineral material, said ingredients being as defined herein, combining said ingredients together; and mixing said ingredients to obtain a homogenous mixture.

Thus, the compositions according to some embodiments of the present invention comprise at least five different ingredients of the eight ingredients. The composition may comprise huang lian, sheng di huang, tian hua fen, shi gao, and at least one more of the eight ingredients. The composition may further comprise shi hu. The composition may comprise fan shi liu ye and at least four more of the eight ingredients. Alternatively or additionally, the composition may comprise wu mei and at least four more of the eight ingredients. Preferably, the composition may comprise huang lian, sheng di huang, tian hua fen, and fan shi lilt ye and/or wu mei. Additionally, the composition may further comprise shi gao and/or shi hu. Preferably, the composition may comprise all of said eight ingredients. Alternatively, the composition may comprise traditional Chinese remedies' ingredients, said ingredients being fan shi liu ye, wu mei, tian hua fen, and shi gao.

### BRIEF DESCRIPTION OF THE FIGURES

FIG. 1 demonstrates the muscle cells viability in presence of the compositions according to an embodiment of the invention.

FIG. 2 demonstrates the muscle cells glucose uptake responsive insulin challenge.

FIG. 3 demonstrates the muscle cells glucose uptake in presence of high concentration of the compositions according to an embodiment of the invention and the prior art compositions.

FIG. 4 demonstrates the muscle cells glucose uptake in presence of low concentration of the compositions according to an embodiment of the invention and the prior art compositions.

### DETAILED DESCRIPTION

The invention relates to compositions that may be useful for treatment of conditions, for example, hyperglycemia, e.g. for reducing high blood sugar levels. The invention also relates to methods of making such compositions. The invention also further relates to methods of treatment using such compositions.

As discussed in the background section above, excess blood sugar, for example in mammals, is considered a medical problem. Hyperglycemia is the hallmark symptom of various ailments, mainly, diabetes and pre-diabetic conditions. Diabetes mellitus, usually referred to as just diabetes, is a group of metabolic disorders characterized by a high blood sugar concentrations over prolonged time intervals. Symptoms often include frequent urination due to glycosuria, and thus increased thirst and increased appetite. Untreated, diabetes can cause many health complications, including diabetic ketoacidosis, hyperosmolar hyperglycemic state, and even death. Serious long-term complications include cardiovascular disease, stroke, chronic kidney disease, foot ulcers, neuropathy, ophthalmopathy, including retinopathy, and cognitive impairment. Diabetes symptoms occur when the pancreas is not producing enough insulin, and/or when the cells of the body are not responding properly to the insulin produced, e.g. by not uptaking glucose from the blood, etc.

There are three main types of diabetes mellitus. So-called “Type 1 diabetes”, also referred to as “insulin-dependent diabetes mellitus” or “juvenile diabetes”, occurs due to loss of beta cell in Langerhans islets in pancreas, which results in failure of the pancreas to produce enough insulin. The loss of beta cells is usually caused by an idiopathic autoimmune reaction against Langerhans islets. Other type of diabetes is “Type 2 diabetes”, which is not caused by the loss of insulin-producing cells, but rather by the peripheral cells not responding properly to insulin signaling. This form is also known as “non-insulin-dependent diabetes mellitus” or “adult diabetes”. The hallmark of type 2 diabetes is the insulin resistance, a condition in which peripheral cells become insensitive to changes of insulin concentrations. As the disease progresses, the insulin-producing cells are overstimulated, producing hyperinsulinemia, and as a result of this stress, loss of the beta cells may occur later as the disease progresses, which will warrant exogenous insulin administration. Another type of diabetes is gestational diabetes, and occurs sometimes in pregnant women without a previous history of hyperglycemia. Gestational diabetes usually resolves after the birth of the baby. Prolonged elevated concentrations of glucose in the blood usually results in glycation of hemoglobin, resulting in the adduct of hemoglobin and a sugar, usually glucose, fructose, or galactose, called “hemoglobin A1C”, or just HbA_{1C}. Glycated hemoglobin has been used to estimate the average glucose levels retroactively. For example, the HbA_{1C} value of 5 indicates estimated average glucose concentration as 97 mg/dL; HbA_{1C} value of 6 indicates estimated average glucose concentration as 126 mg/dL; and HbA_{1C} value of 7 indicates estimated average glucose concentration as 154 mg/dL.

Human normoglycemia, i.e. the normal blood glucose concentrations, is presently defined as fasting glucose of less than 100 mg/dL, and postprandial glucose concentration two hours after a normalized glucose tolerance test of below 140 mg/dL. These values are updated from time to time by various authorities, as more scientific evidence emerges. Glucose intolerance (or so-called “pre-diabetes”) is usually defined as fasting glucose concentration between 100-125 mg/dl (or between 140 and 200 mg/dL after a glucose tolerance test), and Type-2 diabetes is typically diagnosed in humans having a blood glucose concentrations higher than 126 mg/dL in fasting state, and above 200 mg/dL after glucose tolerance test. Additionally, glycated hemoglobin may be used to diagnose diabetes and pre-diabetic conditions. Thus, HbA_{1C} value of 6.5 and above is usually considered to indicate diabetes, with the estimated average glucose concentration of 140 mg/dL. However, vigorously reducing the HbA_{1C} value to below 6 is not recommended due to increased mortality in diabetic patients, and HbA_{1C} values of between 6.5 and 7 is considered a reasonable target of treatment in type 2 diabetic patients.

As mentioned above, under traditional Chinese practice, the symptoms of diabetes and pre-diabetic conditions (even if initially asymptomatic) usually correspond to the “wasting and thirsting disorder”, or xiao ke ( [symbol] ). The diagnosis is usually made according to traditional diagnostic questioning. The questions are related to the organ interrelationships, such as lung/stomach (sheng cycle), spleen/stomach (internal/external pairing), and spleen/kidney (ke cycle). The symptoms of wasting, tongue and pulse that are taken into consideration include red tongue edges and tip, thin yellow tongue fur, surging rapid pulse (so-called upper wasting), or red tongue, yellow fur, slippery large forceful pulse (middle wasting); or red tongue with scanty yellow fur or scanty fluids, and fine wiry pulse (lower wasting). The symptoms of appetite, thirst, and urinary symptoms include thirst with excess drinking, frequent excessive urination (upper wasting: lung/stomach heat/fire, fluid damage, correlates with early-stage diabetes); big appetite and rapid hungering, lesser thirst and polyuria, emaciation (middle wasting: stomach fire); excess thirst, frequent copious cloudy urination, nocturia (qi/yin deficiency); frequent excessive possibly turbid urination, more frequent at night (lower wasting: kidney yin deficiency); excess thirst, normal/reduced appetite (spleen/stomach qi deficiency); thirst with profuse drinking, excessive eating, rapid hungering (damp heat); reduced polydipsia/polyphagia/polyuria, frequent, clear, sometimes profuse urination or urinary incontinence (spleen/kidney yang deficiency). The aetiology is also considered: kidney essence deficiency (juvenile onset); ageing, unregulated eating and drinking (adult onset); and emotions (qi stagnation and blood stasis as underlying patterns). Thus, xiao ke is considered to involve the following zangfu patterns:
Lung/stomach heat/fire with fluid damage;
Stomach heat/fire;
Qi and yin deficiency;
Kidney yin deficiency;
Spleen and stomach qi deficiency;
Damp heat obstructing the middle burner; and
Spleen/kidney yang deficiency.

Xiao ke is divided into four stages, with the fifth stage being split to a variety of complications. During the first stage the patient is unrestrainedly feeding on rich, sticky, sweet, and high-protein food. During the second stage the patient may still be asymptomatic, whereas a general weakening of the gastrointestinal tract and the lungs may be observed. These stages roughly correspond to pre-diabetic conditions.

However, under modern holistic practice, measurement of blood glucose, fasted or after a glucose challenge, and glycated hemoglobin, may be used as auxiliary tool to determine xiao ke.

As can be readily seen in the Table B above, despite the very different composition of Qin Wei San and Bai Hu Tang, there are several remedies that they share in common. These include huang lian, sheng di huan, tian hua feng, and an optional component of QWS shi gao. It has now been unexpectedly found that combinations of certain four herbal remedies could be equipotent, under the tested conditions, to the traditional preparations Qin Wei San and Bai Hu Tang, in terms of glucose uptake by the muscle cells. Moreover, the combination of eight herbal remedies can be superior to either of the traditional remedies, and with at least similar efficacy but with increased potency, e.g. requiring lesser dose to achieve the same effect.

Thus, in a first aspect of the present invention, disclosed herein are useful compositions of herbal components, e.g. in form of traditional Chinese remedies, that, when administered to a patient in need thereof, e.g. a human or a non-human mammal, have a beneficial effect. Usually, upon administration of the composition to the patient reduces high blood sugar levels. As a result, such compositions are useful for reducing high blood sugar levels. Whereas the exact mechanism of action of the composition or of the components thereof is not knows or material, without being bound by a particular theory it is believed that the administration of the composition may directly reduce the blood sugar level, e.g. via induced uptake of glucose to the body cells, and/or by amending the disrupted pathways in the cells that cause them to react inadequately to insulin, i.e. via treating the underlying pathology that leads to the high blood sugar level. The experimental data demonstrated in the examples shows the biological effect on glucose uptake by the muscle cells, the main glucose-consuming cells in the body. It has been surprisingly found that combining specific herbal components it may be possible to achieve an improved glucose uptake, relatively to existing compositions.

In some embodiments, the compositions according to the invention may be useful for reducing the blood sugar level of a subject to which the composition is administered. Preferably, the subject is a human, or a non-human animal, in particular a non-human mammal.

The composition may be useful for treating hyperglycemia-associated conditions, such as pre-diabetes, diabetes (especially Type II diabetes), and gestational diabetes. The composition may be administered to prevent the ailment, i.e., the composition may be administered prophylactically to prevent the development of an ailment, particularly in subjects falling under one or more risk factors for developing diabetes. The composition may be administered to stop the progression of the disease, or to reduce the rate of progression of the disease.

Thus, the compositions of the teachings herein comprise at least five different ingredients of the eight ingredients. These eight ingredients may be grouped into four components as follows. The first component is Psidium guajava L. ( [symbol] , fan shi hu ye). The second component includes Herba dendrobii ( [symbol] , shi hu) and/or Mume fructus ( [symbol] , wu mei). The third component includes Radix trichosanthis kirilowii ( [symbol] , tian hua fen) and/or Gypsum ( [symbol] , shi gao). The fourth component includes at least two ingredients of Rhizoma coptidis ( [symbol] , huang lian), Radix rehmanniae glutinosae ( [symbol] , sheng di huang), and Radix et Rhizoma rhei ( [symbol] , da huang). Although the preferred compositions comprise all eight ingredients, these components demonstrate the principles of compounding the composition. For example, when either the component iii) or iv) comprises all its ingredients, the other component is optional i.e. when the composition comprises tian hua fen and shi gao, neither huang lian, sheng di huang, nor da huang is required, and conversely, when the composition contains huang lian, sheng di huang, and da huang, no tian hua fen nor shi gao is required.

According to further principles, sometimes the components may be substituted for other components, with similar traditional action, but with increased or decreased potency. For example, huang lian may be at least partially substituted for huang qin [ [symbol] ] (Radix scutellariae) in a dose of 5-20% wt, or for ku gua [ [symbol] ] (Momordica charantia), in a dose of 3-30 weight percent. Sheng di huang may be at least partially substituted for shu di huang [ [symbol] ] Radix rehmanniae Preparata in a dose of 5-30% wt, or for tian men dong [ [symbol] ] Asparagi radix), in amounts of up to 40 weight percent. Da huang may be at least partially substituted for Hu Zhang [ [symbol] ] Rhizoma polygoni cuspidati in amounts between 5-20 weight percent. Shi hu may be at least partially substituted for ge gen [ [symbol] ] (Radix puerariae) in a dose of 3-15% wt, for Han lian cao [ [symbol] ] (Herba ecliptae prostratae), at 5-20% wt, or for Mai men dong [ [symbol] ] (Ophiopogonis radix) at 5-30% wt, or for Hu Lu Ba [ [symbol] ] (Semen trigonellae), at 1-20% wt. Shi gao may be at least partially substituted for tian men dong [ [symbol] ] Asparagi radix), at 5-30% % wt, or for Da Qing Ye [ [symbol] ] (Folium istadis), at a dose of 5-40% wt. Tian hua fen may be at least partially substituted for zhi Mu [ [symbol] ] (Rhizoma anemarrhenae) in amounts between 5-30% wt. or for Shan Yao [ [symbol] ] (Rhizoma dioscoreae), in a dose of 5-30% wt. Wu mei may be at least partially substituted for Wu Wei Zi [ [symbol] ] (Fructus schisandrae), in an amount of 1-20% wt. Fan shi liu ye may be at least partially substituted for Gan Lan Ye [ [symbol] ] (Olea europaea), in an amount of 5-30% wt, or for shi geng teng [ [symbol] ] (Gymnema sylvestre, known also as gurmar) 5-40% wt, or for Ku Gua [ [symbol] ] (Momordica charantia), in a dose of 3-30% wt.

As mentioned above, the term “eight ingredients” as used herein refers to the list of components of the Table 1, unless the context clearly dictates otherwise.

Thus, according to an aspect of the present invention, provided herein a composition comprising a combination of at least five ingredients selected from the group of eight ingredients consisting of: Rhizoma coptidis ( [symbol] , Huang Lian), Radix rehmanniae glutinosae ( [symbol] , Sheng Di Huang), Gypsum ( [symbol] , Shi Gao), Radix et Rhizoma rhei ( [symbol] , Da Huang), Herba dendrobii ( [symbol] , Shi Hu), Mume fructus ( [symbol] , Wu Mei), Radix trichosanthis kirilowii ( [symbol] , Tian Hua Fen), and Psidium guajava L. ( [symbol] , Fan Shi Liu Ye). In some further embodiments, the composition comprises six ingredients selected from the group of eight ingredients. In some additionally further embodiments, the composition comprises seven ingredients selected from the group of eight ingredients. In some embodiments, the composition comprises all eight ingredients.

TABLE 1

| | Chinese | Pin Yin | |
|---|---|---|---|
| Botanical Name | Name | Name | Traditional action |
| Rhizoma Coptidis | [symbol] | huang lian | Clears and drains Fire and |
| | | | drains Dampness |
| Radix Rehmanniae | [symbol] | sheng di | Clears Heat, cools blood, and |
| Glutinosae | | huang | nourishes Yin |
| Radix et Rhizoma | [symbol] | da huang | Drains Heat and Fire and |
| Rhei | | | purges accumulations |
| Herba Dendrobii | [symbol] | shi hu | Nourishes Yin, clears and |
| | | | reduced Heat, augments Jing |
| Gypsum | [symbol] | shi gao | Clears Heat from Qi, drains |
| | | | and clears Fire |
| Radix Trichosanthis | [symbol] | tian hua | Drains Heat, moistens Lung |
| Kirilowii | | fen | Dryness, generated fluids |
| Mume Fructus | [symbol] | wu mei | Stops leakage of Qi, generates |
| | | | fluids |
| Psidium guajava L. | [symbol] | fan shi | Dry dampness and benefit the |
| | | liu ye | spleen, digestive system, clear |
| | | | heat and toxin (out of body) |

Additionally, the composition may comprise at least one of the following ingredients: Rhizoma anemarrhenae asphodeloidis ( [symbol] , zhi mu), Cortex mouton radicis ( [symbol] , mu dan pi); Crataegi fructus ( [symbol] , shan zha), Penthorum chinense Pursh Flower ( [symbol] , gan huang cao); and Silybum marianum ( [symbol] , shui fei ji).

These ingredients typically comprise between 60 and 100% by weight of the composition, e.g. between 65 and 100%, or between 70 and 100%, or between 75 and 100%, or between 80 and 100%. Within the totality of these ingredients, the composition may comprise between 7 and 14% wt huang lian, between 4 and 12% wt sheng di huang, between 2 and 8% wt da huang, between 4 and 12% wt shi hu, between 2 and 8% wt shi gao, between 7 and 14% wt tian hua fen, between 7 and 14% wt wu mei, and between 37 and 45% wt fan shi liu ye. Further, the composition may comprise between 1 and 15% wt huang lian, between 5 and 40% wt sheng di huang, between 2 and 15% wt da huang, between 4 and 20% wt shi hu, between 2 and 30% wt shi gao, between 5 and 35% wt tian hua fen, between 5 and 20% wt wu mei, between 5 and 50% wt fan shi liu ye, between 5 and 30% wt zhi mu, between 3 and 20% wt mu dan pi, between 4 and 20% wt shan zha, between 5 and 30% wt gan huang cao, and between 3 and 30% wt shui fen ji.

The composition may comprise all eight ingredients. When the composition comprises all of the eight ingredients, without being bound by any particular theory it is believed that the composition is the most balanced and suitable for long-term treatment, without excessive effects on any of the traditional axes. As demonstrated in the Examples below, the composition could be successfully used for extended time intervals; however, when other compositions were used, including fan shi liu ye alone, the treatment could not be well tolerated. For instance, in the formula of all of the eight components, the presence of wu mei is believed to balance the effects of other ingredients, and to improve the overall “taste” component of the formula (the term “taste” component referred to in this context is as known in the traditional Chinese medicine, alongside the “temperature” of the ingredient and the affected “meridians”). It is also believed that the presence of wu mei (and shan zha) in the formula could assist in supporting the liver, via its effect on liver meridian, and thus promote the beneficial effect that the liver has on regulation of glucose blood concentrations. Additionally, when the composition comprises the preponderance of “bitter” and “cold” ingredients, the stool consistency could be decreased and cause diarrhea in long term, and thus the addition of wu mei could be beneficial in this aspect as well. The inclusion of shi gao, on the other hand, that expels heat from the stomach and may assist in the overall effect via reduction of hunger, when unbalanced, might cause also the decrease in digestion that occurs therein. This effect can slow down the food degradation (the beneficial effect), but in the long term may exhaust the digestive system, if not properly balanced.

Additionally, Huang Lian (Rhizoma coptidis) and Shi Gao (Gypsum) clear heat and drain fire, clears blazing stomach fire, clear toxin out of the body (in stool), treat ravenous hunger, and help lowering the blood sugar levels. With Sheng Di Huang (Rehmanniae radix) being very sticky, assistant to HL clear heat injuring the nutritive level. Also, with the addition of Tian Hua Fen (Trichosanthis radix), these two herbs SDH and THF are main to balance HL that can be very dry, moisturize the body and specially the stomach and help to reduce thirst and hunger. Da Huang (Radix et Rhizoma rhei) and Fan Shi Liu Ye (Psidium guajava L.) are both are active the large intestine and assist to bowl movement, lowering stool, clear toxics and move out of the body. Wu Mei (Mume fructus) balance HL and SG and assistant SDH THF to moist the body by strong effect of generating fluid. Shi Hu (Dendrobii herba) take to position in the middle level of action, clears heat and generate fluids, therefore enriches the kidneys, yin that nourishes Stomach Yin, augments the essence, brightens vision and strengthens the lower back (Tonifies Kidneys that damaged in diabetic condition reduces heat deficiency).

Whereas the compositions preferred are the balanced compositions suitable for the long-term treatment, e.g. the all eight ingredients' composition, other compositions are included within the scope of the present invention, which could be used for short and long term management of hyperglycemia. For example, the composition may preferably comprise the combination of huang lian, sheng di huang, da huang, shi hu, and at least one more of the eight ingredients of the Table 1. Alternatively, the composition may also preferably comprise shi gao, tian hua fen, wu mei, fan shi hu ye, and at least one more of the eight ingredients. The composition may further preferably comprise huang lian, sheng di huang, tian hua fen, shi gao, and at least one more of the eight ingredients. In some particularly preferred embodiments, the composition may comprise huang lian, sheng di huang, tian hua fen, shi gao, shi hu, and at least one more of the eight ingredients.

Preferably, the composition comprises fan shi hu ye and at least four more of the eight ingredients. Additionally or alternatively, the composition comprises wu mei and at least four more of the eight ingredients. Specifically, the composition may comprise fan shi hu ye and/or wu mei, and a combination of huang lian, sheng di huang, tian hua fen, and optionally shi gao and/or shi hu. Preferably, the composition may comprise fan shi hu ye and wu mei, and a combination of huang lian, sheng di huang, tian hua fen, and optionally shi gao and/or shi hu.

The compositions may further comprise additional suitable herbs or minerals e.g., to accommodate specific personal traits, as known in the Chinese medicine. The additional components may be sometimes selected from the components enumerated in the abovementioned Xie publication.

The relative amounts of the ingredients are any suitable relative amount. Generally, the relative amounts are such that the amount of each of the ingredients between 1.5% and 70% by weight of the composition, provided that the total sums up to 100%. The relative amounts of each ingredient will be dependent on the ingredient potency and collaborative traditional action within the combination, as described above.

Additionally, Zhi Mu (Rhizoma anemarrhenae asphodeloidis) may be used to ameliorate the dryness of tonifying or warming substances. Assistant to SH to generate fluids, but stronger in clearing heat and draining the stomach fire, therefore assisting THF to reduce thirst and hunger. Shan Zha (Crataegus fructus) may relieve food stagnation and treats hypertension, transform blood stasis and dissipate clumps due to blood stasis. It may treats accumulation of meats or greasy foods. This herb is sour as WM but moderate, therefore, assist to WM to moisten the body by effect of generating fluid. ZM and SZ are warm herbs and may be used to balance the nature in the formula.

Mu Dan Pi (Cortex moutan radicis) clears ascending liver fire, clears fire from deficiency, clears heat and cools the blood, drains pus and reduces swelling, invigorates blood and dispels blood stasis, with DH it is normally used for abdominal pain, constipation, and low-grade fever associated improve clear toxin in the liver organ and drain out by stools with unsupported intestinal abscess as in “Da Huang Mu Dan Tang” (classic formula). With ZM and SDH it is for night fevers and recurrent afternoon fevers due to heat lingering at the nutritive level in warm-heat pathogen diseases, as in Qing Hao Bie Jia Tang (classic formula). Gan Huang Cao (Penthorum chinense Pursh Flower) and/or Shui Fei Ji (Silybum marianum). May be used to balance HL, SG, DH and to protect the liver organ in a long time use of this formula.

Generally, as demonstrated in the Tables 2a-2e below, the components, if present, may be present in following weight percentage parts in the composition, depending on whether other components are present or absent, the weight percentage of the components in the composition may vary significantly.

TABLE 2a

| Botanical Name | Pin Yin Name | % wt |
|---|---|---|
| Rhizoma Coptidis | huang lian | 3-15 |
| Radix Rehmanniae Glutinosae | sheng di huang | 10-40 |
| Radix et Rhizoma Rhei | da huang | 2-15 |
| Herba Dendrobii | shi hu | 5-20 |
| Other ingredients | | 10-67 |

TABLE 2b

| Botanical Name | Pin Yin Name | % wt |
|---|---|---|
| Gypsum | shi gao | 5-30 |
| Radix Trichosanthis Kirilowii | tian hua fen | 10-35 |
| Mume Fructus | wu mei | 5-25 |
| Psidium guajava | fan shi liu ye | 10-45 |
| Other ingredients | | 10-42 |

TABLE 2c

| Botanical Name | Pin Yin Name | % wt | |
|---|---|---|---|
| Rhizoma Coptidis | huang lian | 10-35 | 3-15 |
| Radix Rehmanniae Glutinosae | sheng di huang | 5-15 | 20-40 |
| Radix Trichosanthis Kirilowii | tian hua fen | 15-35 | 5-20 |
| Gypsum | shi gao | 15-30 | 2-10 |

| | | |
|---|---|---|
| Herba Dendrobii | shi hu | 5-20 |

| | | |
|---|---|---|
| Other ingredients excl. fan shi liu ye | 10-55 | |
| Other ingredients incl. fan shi liu ye | | 10-65 |

TABLE 2d

| Botanical Name | Pin Yin Name | % wt | | | | | |
|---|---|---|---|---|---|---|---|
| Psidium guajava leaf | fan shi liu ye | 15-45 | | 15-45 | 15-45 | | 15-45 |
| Mume Fructus | wu mei | | 5-20 | 5-20 | | 5-20 | 5-20 |
| Rhizoma Coptidis | huang lian | 3-15 | 3-15 | 3-15 | 3-15 | 3-15 | 3-15 |
| Radix Rehmanniae Glutinosae | sheng di huang | 5-15 | 15-40 | 5-40 | 5-15 | 15-40 | 5-20 |
| Radix Trichosanthis Kirilowii | tian hua fen | 5-20 | 5-35 | 5-35 | 5-20 | 5-35 | 5-35 |
| Herba Dendrobii | shi hu | | | | 5-20 | 5-20 | 5-20 |
| Gypsum | shi gao | | | | 2-10 | 15-30 | 2-10 |
| Other ingredients | | 5-72 | 2-72 | 2-67 | 2-65 | 2-52 | 2-60 |

TABLE 2e

| Botanical Name | Pin Yin Name | % wt | % wt |
|---|---|---|---|
| Rhizoma Coptidis | huang lian | 7-14 | 1-15 |
| Radix Rehmanniae Glutinosae | sheng di huang | 4-12 | 5-40 |
| Radix et Rhizoma Rhei | da huang | 2-8 | 2-15 |
| Herba Dendrobii | shi hu | 4-12 | 5-20 |
| Gypsum | shi gao | 2-8 | 2-30 |
| Radix Trichosanthis Kirilowii | tian hua fen | 7-14 | 5-35 |
| Mume Fructus | wu mei | 7-14 | 5-20 |
| Psidium guajava | fan shi liu ye | 37-45 | 5-50 |
| Rhizoma Anemarrhenae Asphodeloidis | zhi mu | | 5-30 |
| Cortex Moutan Radicis | mu dan pi | | 3-20 |
| Crataegi Fructus | shan zha | | 4-20 |
| Penthorum Chinense | gan huang cao | | 5-30 |
| Silybum marianum | shui fei ji | | 3-30 |

Additionally, some components may be substituted for a combination of components, or, conversely, several of the eight ingredients may be substituted for a single ingredient. For example: rou gui [ [symbol] ] cinnamon (3-30% wt), zhi gan cao[ [symbol] ] Radix glycyrrhizae preparata (3-15% wt), Hu Lu Ba [ [symbol] ] Semen trigonellae (3-20% wt), ku gua [ [symbol] [symbol] ] Momordica charantia (3-30% wt), lu cha [ [symbol] ] green tea (5-30% wt), shi geng teng [ [symbol] ] (Gymnema sylvestre, known also as gurmar) 5-40% wt, gan lan ye [ [symbol] ] Olea europaea (5-30% wt), or dan shen [ [symbol] ] Salviae miltiorrhizae radix (5-20% wt), may all be used in addition to or instead of at least a part of some of the components.

The Chinese traditional remedies usually refer to botanical or mineral component, but the traditional names also usually refer to the particular way these botanical or mineral components are obtained and prepared. Apart from the specific botanical part of a botanical remedy, the name usually also implies that the component was harvested from a specific plant, the material has been obtained from a specific plant organ, was audited for the classical description of appearance, color, taste, odor, and other traditional tests, and then optionally prepared according to the traditional way, such as dried and ground, boiled in water, cooked in honey, or otherwise modified according to the Chinese medical tradition. Similarly, mineral remedies are audited for the appearance, color, particle size, etc. Therefore, most preferably, the names of all traditional Chinese remedies refer to the “compendial” forms of the remedies, prepared according to the rules of the traditional Chinese medical compounding. Nevertheless, in some embodiments, the herbal components of the compositions as described herein may be present in conventional herbal form as known in the Western medicine, i.e. as an aqueous or organic extract, tincture, dried extracts, standardized extracts, and the like. Each of the components may be present in a suitable form, and some of the components of the composition may be present, e.g. as aqueous extract, whereas some others may be in traditional forms, or as dried organic extract, etc.

The composition any further comprise any pertinent amount of inactive components, e.g. inert excipients, which may be used to facilitate formulating the composition to a suitable administrable form, e.g. as described below. Particularly, a composition may be adapted for oral administration.

The composition may usually be a pharmaceutical composition or a nutraceutical composition. The composition may also be provided in a form of a food additive or a dietary supplement. The composition may preferably be provided as an edible article, being a dosage form for the compositions as disclosed herein, for the purposes of the present disclosure.

When the composition is provided in a comestible form, e.g. as an edible (comestible) article, it may be contained in any suitable edible form, but preferably the edible article is a snack or a dessert. Preferably, the comestible articles are provided as individual portions of edible articles, e.g. comprising a single dose (e.g. unit dose) of the composition. The suitable form of individual portions may include chocolate bars, e.g. pre-split bars, chocolate cushions, individually wrapped candy, energy bars, cookies, cake portions, crackers, pastilles, gums, films, and the like. The composition may also be provided in an edible spread, e.g. in single-dose squeezable tubes, single-serving packets, and the like. The composition may also be provided as a multi-dose container and/or dispenser.

As described below, the usual dose of the composition may be dependent on the components of the composition, but may usually be between 500 mg and 15 grams, particularly when the composition is provided as a mixture of the traditionally compounded remedies. However, when the composition is provided as dried extracts of the composition or of the individual components thereof, the dose may be significantly reduced in some instances. However, it may be advantageous to utilize traditionally compounded remedies nevertheless; it is currently believed without being bound by a theory that the taste of the traditional remedies, particularly incorporated into edible articles, may contribute to the palatability of the edible articles and to the individual feeling of the healthy food, apart from advantageous conforming to the traditional definitions of “natural” products. The preferred single dose of the composition may be between 1 and 3 grams.

Although the composition is preferably a new traditional Chinese formula, in some embodiments the composition may further comprise a pharmaceutically active ingredient. In these embodiments, the active pharmaceutical ingredient may be selected from conventional antidiabetic drugs, such as sulfonylureas, thiazolidinediones, and peptidyl peptidase IV inhibitors. The dose of the composition will then be determined by the dose of the pharmaceutically active ingredient, and its weight ratio in the composition.

When the composition is provided as an edible article, it may comprise conventional culinary components as known in the art. Particularly, when the edible article is a chocolate, it may comprise an edible fatty carrier, e.g. cocoa butter, and also cocoa mass, milk or milk components, a syrup, caramel, nuts and/or seeds, and the like. The edible article may further comprise the anti-oxidants, coloring agents, gelling agents, thickening agents, stabilizers, preservatives, and the like.

Preferably, the edible article is a low-sugar or no-sugar article, e.g. containing little carbohydrates with high glycemic index. Preferably, the edible article contains little nutritive sugars. Preferably, the edible article contains no sucrose or glucose, and further preferably no fructose or maltose. The edible article may contain sweeteners, including compounds with some nutritive value, like some of polyols or low-calorie carbohydrates, such as allulose. The sweeteners that may be used include polyols, e.g. erythritol, isomalt, maltitol, mannitol, sorbitol, and xylitol. The sweeteners may be artificial sweeteners, e.g. salts of aspartame, saccharin, acesulfame (preferably potassium acesulfame), and also sucralose or cyclamate. Further sweeteners may include steviol glycosides, e.g. stevia plant products. Additionally, the edible article may contain monk fruit extract, also known as Swingle fruit extract (Siraitia grosvenorii, or [symbol] —luo han guo in Chinese terms).

The suitable additives may also be from the lists of regulatory bodies, e.g. from the Substances Added to Food inventory (previously known as Everything Added to Foods in the United States—EAFUS). For further compositions of the edible articles and for the processes of the manufacturing thereof, reputable references may be consulted, e.g. Handbook of Food Products Manufacturing, published on 1 Aug. 2006, edited by Y. N. Hui, ISBN: 978-0-470-04964-8, doi: 10.1002/0470113553.

A composition according to the teachings herein may be a pharmaceutical composition. The suitable pharmaceutical dosage forms of the composition are preferably oral administrable dosage form. These include a liquid, e.g. a solution comprising soluble extracts of the composition components, a tincture of the composition components, a dispersion of the soluble and insoluble composition components, and an elixir. Preferably, the dosage form is a solid dosage form, e.g. a tablet or a capsule, comprising the solid components of the composition. The dosage form may also be in a form of a gel-cap, e.g. comprising oil-soluble/dispersible components of the composition. The pharmaceutical compositions are preferably also sugar-free, and may contain non-caloric sweeteners, if required.

Preferably, the dosage form is a capsule. The capsule may be filled with the composition of the formula components, homogenously blended together. The filling blend may then further comprise a filler, e.g. lactose or microcrystalline cellulose, a lubricant, e.g. a fatty acid or its salt, like magnesium stearate, a disintegrant, e.g. crosscarmelose, crosspovidone, or sodium starch glycolate, a glidant, e.g. colloidal silicon dioxide (or talc, which is hua shi [symbol] —one of the components of FFTSS traditional formula, vide supra), an antioxidant, an antimicrobial preservative, a colorant, and/or a fragrance. The components may be granulated, e.g. to improve the flow properties of the powder. In these embodiments the composition may further comprise a binder, e.g. a povidone, a hypromellose, a methylcellulose, a hydroxypropyl cellulose, or an ethyl cellulose. The capsule may then be coated with an immediate-release sealing coating, e.g. of Opadry™ family, such as hypromellose or polyvinyl alcohol based coating.

The composition and a dosage form as described herein may be made using processes well known in the art, e.g., by conventional pharmaceutical and food technology processes. Some of these techniques are described, inter alia, in Remington: The Science and Practice of Pharmacy, by Pharmaceutical Press (ISBN 978-0-85711-062-6), also known as “Remington's Pharmaceutical Sciences”.

Generally, the compositions are manufactured by combining together the components of the composition. Dosage forms and edible articles are then manufactured by combining the composition with the components of the dosage forms or the recipe components of the edible articles.

The components are usually weighed with the required accuracy, e.g. depending on the scale, and mixed together. The components may be first ground to a fine powder prior to combining with other components. Alternatively, the weighed components may be advantageously ground together, e.g. with a blade mill, to provide a homogenous fine powder of the composition. The grinding may usually be performed as known in the art, e.g. using a suitable mill. The mill may be a blade mill, a rotor mill, a hammer mill, a ball mill, and the like. Blending may be carried out using a variety of equipment, e.g. tumblers, mixers, for a time sufficient to produce a homogenous blend. When the composition is provided into an edible article, it may be combined first with a liquid that is present in the recipe, e.g. an oil. The composition may be granulated, if particles with uniform size larger than e.g. 50 microns may be required, for example for capsule filling. The granulation may be performed using conventional granulators, e.g. high-shear granulators. Binders and/or granulation liquid may be added to the blend during the granulation. The wet granulation may then be dried, e.g. in tray oven, or in a fluid bed drier. The dried granulation may then be further ground using a mill to a desired particle size. The additional components may then be added and blended, as described above. When the composition is provided into a chocolate product, it may be blended with cocoa butter at an elevated temperature, e.g. above the melting temperature of cocoa butter. Other components may then be added, including a sweetener. The chocolate mass may then be poured into molds of suitable size, and cooled to solidification. The composition may also be provided in a suitable carrier into the filling of chocolate items (e.g. pralines). The chocolate forms may then be prepared as known in the art, e.g. by wetting the base mold with the chocolate, letting it run off and cooling it to solidify; the filling may then be applied into the chocolate forms, the filling comprising the blend of the composition, and the sealing layer of the chocolate may then be applied, and cooled to solidify.

Various other features according to the invention as described herein for the aspect of compositions and/or dosage forms are applicable mutatis mutandis to the methods of manufacturing of the composition according to the teachings herein.

The composition may be used for treating a subject in need thereof. Particularly, the compositions may be used to treat a subject suffering from hyperglycemia-related disorder, or a subject who is susceptible to suffering from hyperglycemia, i.e. a subject having one or more of known risk factors for development of hyperglycemia. Preferably, the subject is a human, although in some embodiments the subject may be a non-human animal, in particular a non-human mammal.

In some embodiments, administering the composition comprises administering a dosage form comprising the composition, or administering an edible article comprising the composition. The administering is usually performed according to the desired dose at a desired regimen.

The dosage administered is any suitable dosage and can be determined by a practitioner based on factors such as the weight of the subject and the severity of the condition being treated. It is currently believed that the required dosage for an adult human suffering from pre-diabetes is not less than 500 mg composition, at least twice a day, and even not less than 1 g twice a day. However, as demonstrated in the examples section below, a composition according to the invention may be at least 10 times more potent that, e.g. Bai Hu Tang formula. Therefore the dose required may preferably be lower than required for the traditional Chinese formulae. The daily dose may thus usually be between 500 mg and 10 grams, e.g. between 1.0 and 8 grams, or between 1.2 and 6 grams. The dose may be administered in divided equal or unequal doses, e.g. twice daily, three times daily, four times daily, or more times daily. As shown in the appended examples, the composition may exert anti-diabetic effect by increasing tissue uptake of glucose, therefore in a preferred embodiment the dosage regimen is before or with every meal, but no more than 5-6 times a day. Preferably, the dose is between 500 and 1500 mg, administered 1-4 times a day.

In some such embodiments, the administering by oral administration of the composition to the subject constitutes treating the condition of the subject. Preferably, the treatment leads to a reduction in blood sugar level. In some embodiments, the treatment leads to prevention of the development of an ailment related to high blood sugar levels, e.g., to prevent or reduce the rate of a female subject developing gestational diabetes, to prevent or reduce the rate of a subject developing pre-diabetes, to prevent or reduce the rate of a subject developing diabetes (especially type-2 diabetes). Thus, the composition may be used for prophylaxis of hyperglycemia in a patient in need thereof, i.e. in a patient at risk of developing hyperglycemia, e.g. due to an existing or suspected risk factor.

Various other features according to the invention as described herein for the aspect of compositions and/or dosage forms are applicable mutatis mutandis to the methods of treating hyperglycemia by the composition according to the teachings herein.

### EXAMPLES

### Example 1: Extended Composition

A composition according to the teachings herein was made by providing 100 g of mixture of the twelve ingredients (each ingredient was purchased separately from audited reliable sources in Israel) at the (w/w) percentage as specified in table 3 by mixing the separate ingredients together in a blade blender. The resulting fine homogeneous-looking powder was an embodiment of a composition according to the teachings herein.

TABLE 3

| Ingredient name | % (w/w) |
|---|---|
| Rhizoma Coptidis ( [symbol] , Huang Lian) | 7.5 |
| Radix et Rhizoma Rhei ( [symbol] , Da Huang) | 3 |
| Mume Fructus ( [symbol] , Wu Mei) | 7.5 |
| Radix Trichosanthis Kirilowii ( [symbol] , Tian Hua Fen) | 7.5 |
| Gypsum ( [symbol] , Shi Gao) | 3 |
| Psidium guajava L. ( [symbol] , Fan Shi Liu Ye) | 30 |
| Radix Rehmanniae Glutinosae ( [symbol] , Sheng Di Huang) | 6 |
| Herba Dendrobii ( [symbol] , Shi Hu) | 7.5 |
| Rhizoma Anemarrhenae Asphodeloidis ( [symbol] , Zhi Mu) | 4.5 |
| Cortex Moutan Radicis ( [symbol] , Mu Dan Pi) | 4.5 |
| Crataegi Fructus ( [symbol] , Shan Zha) | 7.5 |
| Penthorum Chinense Pursh Flower ( [symbol] , Gan Huang Cao) | 11.5 |

### Example 2: Preparation of Extended Composition in Chocolate Carrier Unit Doses and Placebo Unit Doses

An edible chocolate mixture was made by combining 75% (w/w) of cacao and cocoa butter mixture and 25% (w/w) of xylitol to obtain an edible chocolate mixture.

An amount of 10% (w/w) of the extended composition of Example 1 was mixed with 90% (w/w) of the edible chocolate mixture and dosage forms according to the teachings herein were made by molding 8 grams of the resulting mixture into an appropriate shape.

Chocolate confections as placebo were made by molding 8 grams of the edible chocolate mixture into an appropriate shape.

### Example 3: Efficacy of the Composition

### Experimental Design

Nine informed human subjects volunteered to test the efficacy of the composition. All the subjects were tested by their primary health care provider as having hemoglobin A1C levels greater than 5.8 DCCT % and had a fasting blood sugar level of at least 100 mg/dl were included in the study. Some of the subjects were regularly taking metformin prescribed by a doctor to treat the prediabetes. Volunteers were guided to test their fasting (or separated from food) glucose levels 6 times a day (glucose levels curve) on a specific schedule (presented as daily average).

All the subjects tested their sugar level at baseline (week 0). For the first week, each subject was instructed to eat 16 control confections a week (about 2 each day). From the second week, each subject was instructed to consume 2 dosage forms a day for between 4 and 9 consecutive weeks. The last glucose levels curve is presented (as daily average) in table 3. In five of the subjects, hemoglobin A1C levels were tested before the first week and tested again three months later.

### Results

The results of the experiment are shown in Table 4 below. From the third and fourth columns of Table 4 it is seen that subjects 1 and 5 showed a substantial reduction in hemoglobin A1C levels. Subjects 2, 4 and 6 showed no substantial change. The hemoglobin A1C levels of subjects 3, 7, 8 and 9 was not tested.

The fifth and sixth columns of Table 4 show the blood sugar level at the start of the experiments and the end of the experiment, respectively. From the seventh column of Table 4, it is seen that of the nine subjects tested, one showed an increase in average blood sugar level (subject 9), one showed no substantial change (subject 8), but the other seven showed a significant decrease of over 10% in average blood sugar level.

TABLE 4

| | | | | | | Change in |
|---|---|---|---|---|---|---|
| | | | | | | blood sugar |
| | | | | Blood sugar | Blood sugar | level |
| | | A1C start | A1C end | level start | level end | [start vs. |
| Subject | Gender | [DCCT %] | [DCCT %] | [mg/dL] | [mg/dL] | end %] |
| 1 | male | 8 | 7.4 | 151 | 124 | −17.7 |
| 2 | male | 6.6 | 6.6 | 113 | 93 | −17.8 |
| 3 | male | — | — | 110 | 99 | −10.3 |
| 4 | male | 6.3 | 6.3 | 133 | 107 | −19.5 |
| 5 | female | 6.5 | 6.1 | 123 | 107 | −12.4 |
| 6 | female | 6.2 | 6.1 | 135 | 103 | −23.5 |
| 7 | male | — | — | 155 | 134 | −13.7 |
| 8 | male | — | — | 141 | 138 | −1.9 |
| 9 | female | — | — | 161 | 177 | 10.2 |

* Start point is referenced as 100%

These results were found to be statistically significant while using one-tailed, paired samples t-test analysis (p<0.05).

### Example 4: Basic Compositions

Additional compositions according to the teachings herein were made by providing 100 g of mixture of ingredients (each ingredient was purchased separately from audited reliable sources in Israel) at the (w/w) percentage as specified in table 5 (each of the columns 4.1, 4.2, 4.3, 4.4, 4.5 and 4.6 refer to different composition), by mixing the separate ingredients together in a blender. The resulting fine homogeneous-looking powders were an embodiment of a compositions according to the teachings herein.

TABLE 5

| | Example | | | | | |
|---|---|---|---|---|---|---|
| | 4.1 | 4.2 | 4.3 | 4.4 | 4.5 | 4.6 |
| Ingredient name | % (w/w) | % (w/w) | % (w/w) | % (w/w) | % (w/w) | % (w/w) |
| Huang Lian | 9 | 12 | 8 | 6 | 5 | 10 |
| Da Huang | | | | | | 4 |
| Wu Mei | | | 15 | | 9 | 10 |
| Tian Hua Fen | 15 | 18 | 32 | 12 | 18 | 10 |
| Shi Gao | 27 | | | 18 | 32 | 4 |
| Fan Shi Liu Ye | | 32 | | 30 | | 42 |
| Sheng Di Huang | 36 | 23 | 27 | 25 | 24 | 8 |
| Shi Hu | 13 | 15 | 18 | 9 | 12 | 10 |

### Example 5: Glucose Uptake by Muscle Cells Responsive to Exposure to the Compositions

The objective of the study was to evaluate the anti-diabetic action of herbal blends. The ability to enhance glucose consumption by skeletal muscle was evaluated in L6 myotubes model in comparison to various control groups, including two known traditional reference formulae.

Cell culturing, materials preparations and all treatments were performed under sterile conditions. The L6 Cells culture medium comprised A-MEM supplemented with 100 U/ml penicillin and 100 μg/ml streptomycin and 10% FBS, sterilely filtered. The L6 cells differentiation medium comprised α-MEM supplemented with 100 U/ml penicillin and 100 μg/ml streptomycin and 2% FBS, sterilely filtered. MTT (3-(4,5-Dimethylthiazol-2-yl)-2,5-Diphenyltetrazolium Bromide) reagents included MTT stock (10×), prepared by dissolving MTT powder in PBS to prepare a 5 mg/ml stock solution. The stock was filtered through 0.2 micron filter, aliquoted and stored at −20° C. At the day of assay, the stock was diluted 1:10 in PBS. The KRBH (Krebs-Ringer-Phosphate-Hepes) buffer contained 20 mM HEPES, 5 mM KH₂PO₄, 1 mM MgSO₄, 1 mM CaCl₂, 136 mM NaCl, and 4.7 mM KCl, had the pH of 7.4. The buffer was prepared freshly in DDW, sterilely filtered. Glucose production buffer was prepared at the day of the experiment, by mixing a fresh solution of PBS containing 2 mM sodium pyruvate, 20 mM sodium lactate, and 100 nM insulin.

The test blends were prepared according to the table 6 below. The numbers are weight used, in grams. The blends were mixed together and ground in a blender, to obtain homogenous mixture of fine powder. The tested items (denoted TI.x, where x is a number between 1 and 3) are as follows. The TI.1 is one of “eight ingredients” compositions, as defined in the description. The TI.2 and TI.3 are four-ingredient compositions, used for comparison.

TABLE 6

| Name | TI.1 - 8HC | TI.2 | TI.3 |
|---|---|---|---|
| huang lian | 1.04 | 1.04 | |
| wu mei | 1.04 | | 1.04 |
| fan shi liu ye | 4.17 | | 4.17 |
| da huang | 0.42 | 0.42 | |
| tian hua fen | 1.04 | | 1.04 |
| shi gao | 0.42 | | 0.42 |
| sheng di huang | 0.83 | 0.83 | |
| shi hu | 1.04 | 1.04 | |
| total | 10 | 3.33 | 6.67 |

The commercial references contained the traditional Bai Hu Tang formula and Qing Wei San, the exact composition whereof is presented in the table 7 below.

For the experiment, the compositions were dissolved in DMSO. For tested article 1 (TI.1), 1 gram of the composition was dissolved in 20 mL of DMSO, to furnish a concentration of 50 mg/mL. For TI.2, 0.33 g were dissolved in 20 mL of DMSO, and for TI.3 0.66 g were dissolved in 20 mL, for comparative reasons, to maintain the same concentration of the ingredients, as in TI.1. BHT and QWS powders were prepared in the same concentration, i.e. 50 mg/mL in DMSO, for testing in the cells. The solutions were diluted with the growth media to the required concentrations.

All the assays were performed in triplicates.

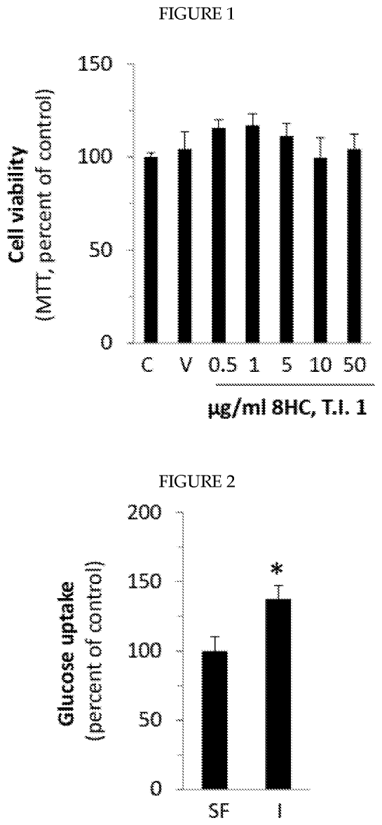

The L6 cells (80,000 cell/ml) were grown and maintained in complete growth media (10% FCS) in tissue flasks at a final volume of 15 ml. The cells were split every other day and maintained at low density. Seven days prior to the experiment, the cells were harvested and seeded in 12 well-plates (1 ml, 80,000 cells per well) at low FCS serum (2%) to induce differentiation. Following differentiation, the cells were treated w/o or with increasing concentrations of TI.1 for 24 hr. In addition, control (naïve) and vehicle (DMSO) control groups were evaluated. The cell viability was monitored by MTT assay. Cells' viability is demonstrated in FIG. 1. In the figure, the cells' viability, expressed as percent of control in MTT test, are shown for the escalating concentrations of TI.1, up to 50 μg/mL. It can be seen that the cells' viability was not affected at all, by either the vehicle (DMSO, labelled “V”), or by either tested concentration of the blend TI.1.

Glucose uptake assay was performed using Glucose Uptake Colorimetric Assay Kit in the non-radioactive 2-deoxy glucose derivative, according to the manufacturers' instructions in KRBH buffer, on differentiated cells. Colorimetric data was quantified by the use of a standard curve. Glucose uptake in the control (naïve cells), vehicle (DDW), and insulin-treated cells was measured. The results are presented in the FIG. 2. It can be readily seen that exposure of the cells to insulin (denoted “I”) has significantly increased glucose uptake by the cells, versus the control (denoted “SF”—serum-free).

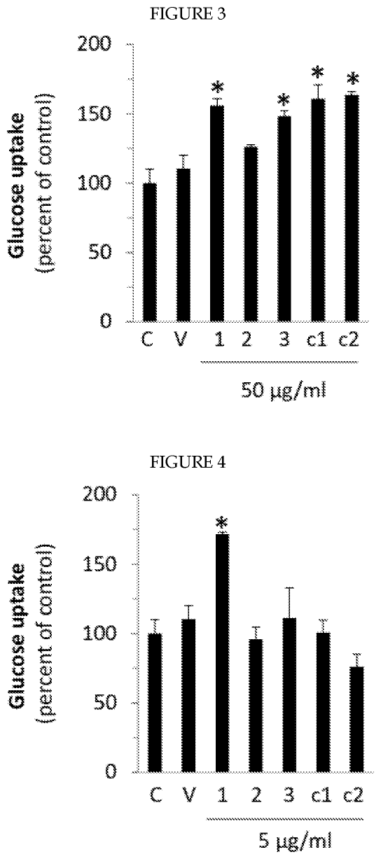

Glucose uptake assay was then performed for the two concentrations of tested items: at 50 μg/mL and at 5 μg/mL. The results are presented in the FIG. 3 and FIG. 4, respectively. It can be readily appreciated from the FIG. 3 that the composition of eight ingredients composition was equipotent to QWS and BHT (denoted as “c1” and c2”) at the concentration of 50 μg/mL. Moreover, TI.3, comprising only four of the ingredients, but including fan shi liu ye and wu mei, was also equipotent to the commercial comparators. However, as readily seen at FIG. 4, when the concentration is reduced to merely 5 μg/mL, the only statistically significant glucose uptake increase was demonstrated with the TI.1, i.e. with the tested “eight-ingredient” composition.

### Example 6—Further Case Studies

Patient 1: a male, 60 years old, suffering from diabetic leg complications and a non-healing wound on the minimus, indicated for amputation. The patient was treated with the composition as in the example 5, TI.1-8HC, 3 grams per day, for 20 days. The treatment was supported with suitable acupuncture protocol, and topical antibiotic preparations. After the treatment, the wound healed completely, the symptoms of the diabetic leg improved, and the patient had stable fasting blood sugar.

Patient 2: a male, 68 years old, suffering from diabetes, a completer of the clinical trial similar to the described in the Example 3, proceeded with the open-label extension for further 3 months and achieved a 24% reduction in the fasting glucose levels. Thereafter the dose was increased to 1500 mg twice daily, and after one month a complete control over fasting glucose level has been achieved (below 140 mg/dL). Low-carbohydrate diets was prescribed to maintain the achievement. The patient presented a year after with fasting blood glucose of over 160; requested and was prescribed raw fan shi liu ye 10000 mg per day (as decoction—10 minutes boiling, chilling, and consumption). After 10 days the fasting blood glucose declined to between 120 and 130 mg/dL, but the patient suffered from fatigue and reverted to the experimental chocolate.

Patient 3: a female, 50 years old, presented during COVID-19 pandemics with fasting blood glucose of 150 mg/dL, H_{A1C} of 7.4, with anxiety and uncontrolled eating. Was prescribed a composition of 500 mg of fan shi liu ye, 500 mg huang lian, and 500 mg shui fei ji, twice daily. After 2 days of treatment the fasting glucose levels dropped to 53 mg/dL, causing hypoglycemia, and the dose was adjusted to once daily. After two months of treatment the fasting glucose blood levels were 105 mg/dL, and was instructed to keep a low-carbohydrate diet to maintain the situation.

Unless otherwise defined, all technical and scientific terms used herein have the same meaning as commonly understood by one of ordinary skill in the art to which the invention pertains. In case of conflict, the specification, including definitions, takes precedence. As used herein, the term “treating” includes curing a condition, treating a condition, preventing a condition, treating symptoms of a condition, curing symptoms of a condition, ameliorating symptoms of a condition, treating effects of a condition, ameliorating effects of a condition, and preventing results of a condition. As used herein, the term “pharmaceutically acceptable” means approved by a regulatory agency of the US Federal or a US state government or listed in the U.S. Pharmacopeia or other generally recognized pharmacopeia for use in animals, and more particularly in humans. Herein, the phrase “pharmaceutically acceptable carrier” refers to an approved carrier or a diluent that does not cause significant irritation to an organism and does not abrogate the biological activity and properties of the administered conjugate. As used herein, the term “carrier” refers to a diluent, adjuvant, excipient, or vehicle with which the therapeutic is administered. Herein the term “excipient” refers to an inert substance added to a pharmaceutical composition to further facilitate processes and administration of the active pharmaceutical ingredients. As used herein, the terms “comprising”, “including”, “having” and grammatical variants thereof are to be taken as specifying the stated features, integers, steps or components but do not preclude the addition of one or more additional features, integers, steps, components or groups thereof. As used herein, the indefinite articles “a” and “an” mean “at least one” or “one or more” unless the context clearly dictates otherwise. As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C). It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements. Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims. Citation or identification of any reference in this application shall not be construed as an admission that such reference is available as prior art to the invention. Section headings are used herein to ease understanding of the specification and should not be construed as necessarily limiting.

1.-16. (canceled)

17. A composition useful for long-term management of hyperglycemia and symptoms thereof, comprising traditional Chinese remedies' ingredients, said ingredients comprise
i) Psidium guajava L. (fan shi liu ye),
ii) Herba dendrobii (shi hu) and/or Mume fructus (we mei),
iii) Radix trichosanthis kirilowii (tian hua fen) and/or Gypsum (shi gao), and
iv) at least two of Rhizoma coptidis (huang lian), Radix rehmanniae glutinosae (shen di huang), and Radix et Rhizoma rhei (da huang),
provided that when either the component iii) or iv) comprises all its ingredients, the other component is optional, and wherein said ingredients comprise between 60 and 100% by weight of the composition.

18. The composition according to claim 17, wherein said composition comprises ii) shi hu and wu mei.

19. The composition according to claim 17, wherein said composition comprises iv) huang lian, sheng di huang, and da huang.

20. The composition according to claim 17, wherein said composition comprises iii) shi gao and tian hua fen.

21. The composition according to claim 17, comprising fan shi lui ye, wu mei, shi hu, tian hua fen, shi gao, huang lian, sheng di huang, and da huang.

22. The composition according to claim 17, further comprising at least one of Rhizoma anemarrhenae asphodeloidis (zhi mu), Cortex moutan radicis (mu dan pi), Crataegi fructus (shan zha), Penthorum chinense (gan huang cao), and Silybum marianum (shui fei ji).

23. The composition according to claim 22, comprising between 7 and 14% wt huang lian, between 4 and 12% wt sheng di huang, between 2 and 8% wt da huang, between 4 and 12% wt shi hu, between 2 and 8% wt shi gao, between 7 and 14% wt tian hua fen, between 7 and 14% wt wu mei, and between 37 and 45% wt fan shi liu ye.

24. The composition according to claim 22, comprising between 1 and 15% wt huang lian, between 5 and 40% t sheng di huang, between 2 and 15% wt da huang, between 4 and 20% wt shi hu, between 2 and 30% wt shi gao, between 5 and 35% wt tian hua fen, between 5 and 20% wt wu mei, between 5 and 50% wt fan shi liu ye, between 5 and 30% wt zhi mu, between 3 and 20% wt mu dan pi, between 4 and 20% wt shan zha, between 5 and 30% wt gan huang cao, and between 3 and 30% wt shui fen ji.

25. The composition according to claim 17, further comprising Siraitia grosvenorii (luo han guo).

26. The composition according to claim 17, wherein said ingredients are provided in any one of following forms: according to traditional Chinese compounding practice, as an aqueous extract, as an organic extract, as a standardized extract, as a tincture, or as a dried extract.

27. The composition according to claim 17, for use in the treating of a condition characterized by hyperglycemia.

28. The composition according to claim 27, wherein said condition comprises pre-diabetes, diabetes, gestational diabetes, glucose intolerance, metabolic syndrome, or xiao ke.

29. The composition according to claim 27, wherein said treating comprises administering to a patient in need thereof of the composition in a dose of between 500 mg and 1500 mg, at a regimen of between once daily and four times daily.

30. The composition according to claim 27, wherein said composition is administered in a form of comestible article.

31. The composition according to claim 30, wherein said comestible article is a portioned edible article, preferably selected from the group consisting of chocolate pre-split bars, chocolate cushions, individually wrapped candy, energy bars, cookies, cake portions, crackers, pastilles, gums, and films.

32. A method of manufacturing of a composition suitable for use in treating a condition associated with hyperglycemia, said method comprising:
a. providing ingredients comprising botanical or mineral material, said ingredients being as defined in claim 17;
b. combining said ingredients together; and
c. mixing said ingredients to obtain a homogenous mixture.
